(19) United States
(12) Patent Application Publication
(10) Pub. No.: US 2003/0034136 A1
(43) Pub. Date: Feb. 20, 2003

(54) Superabsorbent cellulosic fiber

(76) Inventors: Amar N. Neogi, Seattle, WA (US); Richard H. Young SR., Maple Valley, WA (US); Brent A. Petersen, Seattle, WA (US)

Correspondence Address: WEYERHAEUSER COMPANY, INTELLECTUAL PROPERTY DEPT., CH 1J27, P.O. BOX 9777, FEDERAL WAY, WA, 98063, US

(21) Appl. No.: 10/198,814

(22) PCT Filed: Jan. 17, 2001
(86) PCT No.: PCT/US01/01883

(51) Int. Cl. D21C 9/00; D06M 11/56; A61F 13/15; A61F 13/47; A61F 13/49

(52) U.S. Cl. 162/9; 162/157.6; 8/115.51; 8/116.1; 428/906; 428/411.1; 428/537.5; 604/358; 604/375

(57) ABSTRACT

A modified cellulosic fiber having superabsorbent properties is described. The modified fiber of the invention has a fibrous structure substantially identical to the cellulosic fiber from which it is derived. The modified fiber is a water-swellable, water-insoluble fiber that substantially retains its fibrous structure in its expanded, water-swelled state. The modified fiber is a sulfated and crosslinked cellulosic fiber having a liquid absorption capacity of at least about 4 g/g. In one embodiment, the modified fiber is an individual, crosslinked, sulfated cellulosic fiber. In another aspects, the invention provides a rollgood that includes the modified fiber, absorbent composites and articles that include the modified fiber, and methods for making the modified cellulosic fiber.

### FIELD OF THE INVENTION

The present invention relates to a modified cellulosic fiber having superabsorbent properties and, more particularly, to a crosslinked and sulfated cellulosic fiber having a structure substantially identical to the fiber from which it is derived.

### BACKGROUND OF THE INVENTION

Personal care absorbent products, such as infant diapers, adult incontinent pads, and feminine care products, typically contain an absorbent core that includes superabsorbent in a fibrous matrix. Superabsorbents are water-swellable, generally water-insoluble absorbent materials having a liquid absorbent capacity of at least about 10, preferably of about 20, and often up to about 100 times their weight in water. While the core's liquid retention or storage capacity is due in large part to the superabsorbent, the core's fibrous matrix provides the essential functions of liquid wicking, pad strength and integrity, and some amount of absorbency under load. These desirable properties are attributable to the fact that the matrix includes cellulosic fibers, typically wood pulp fluff in fiber form.

For personal care absorbent products, U.S. southern pine fluff pulp is used almost exclusively and is recognized worldwide as the preferred fiber for absorbent products. The preference is based on the fluff pulp's advantageous high fiber length (about 2.8 mm) and its relative ease of processing from a wetlaid pulp sheet to an airlaid web. However, these fluff pulp fibers can absorb only about 2-3 g/g of liquid (e.g., water or bodily fluids) within the fibers' cell walls. Most of the fibers' liquid holding capacity resides in the interstices between fibers. For this reason, a fibrous matrix readily releases acquired liquid on application of pressure. The tendency to release acquired liquid can result in significant skin wetness during use of an absorbent product that includes a core formed exclusively from cellulosic fibers. Such products also tend to leak acquired liquid because liquid is not effectively retained in such a fibrous absorbent core.

The inclusion of absorbent materials in a fibrous matrix and their incorporation into personal care products is known. The incorporation of superabsorbent materials into these products has had the effect of reducing the products' overall bulk while at the same time increasing its liquid absorbent capacity and enhancing skin dryness for the products' wearers.

A variety of materials have been described for use as absorbent materials in personal care products. Included among these materials are natural-based materials such as agar, pectin, gums, carboxyalkyl starch and carboxyalkyl cellulosic fiber, such as carboxymethyl cellulose, as well as synthetic materials such as polyacrylates, polyacrylamides, and hydrolyzed polyacrylonitriles. Although natural-based absorbing materials are well known, these materials have not gained wide usage in personal care products because of their relatively inferior absorbent properties compared to synthetic absorbent materials such as polyacrylates. The relatively high cost of these materials has also precluded their use in consumer absorbent products. Furthermore, many natural-based materials tend to form soft, gelatinous masses when swollen with a liquid. The presence of such gelatinous masses in a product's core tends to limit liquid transport and distribution within the core and prevents subsequent liquid insults from being efficiently and effectively absorbed by the product.

In contrast to the natural-based absorbents, synthetic absorbent materials are generally capable of absorbing large quantities of liquid while maintaining a relatively non-gelatinous form. Synthetic absorbent materials, often referred to as superabsorbent polymers (SAP), have been incorporated into absorbent articles to provide higher absorbency under pressure and higher absorbency per gram of absorbent material. Superabsorbent polymers are generally supplied as particles having a diameter in the range from about 20-800 microns. Due to their high absorbent capacity under load, absorbent products that include superabsorbent polymer particles provide the benefit of skin dryness. Because superabsorbent polymer particles absorb about 30 times their weight in liquid under load, these particles provide the further significant advantages of thinness and wearer comfort. In addition, superabsorbent polymer particles are about half the cost per gram of liquid absorbed under load compared to fluff pulp fibers. For these reasons it is not surprising that there is a growing trend toward higher superabsorbent particle levels and reduced levels of fluff pulp in consumer absorbent products. In fact, some infant diapers include 60 to 70 percent by weight superabsorbent polymer in their liquid storage core. From a cost perspective, a storage core made from 100 percent superabsorbent particles is desirable. However, as noted above, such a core would fail to function satisfactorily due to the absence of any significant liquid wicking and distribution of acquired liquid throughout the core. Furthermore, such a core would also lack strength to retain its wet and/or dry structure, shape, and integrity.

Cellulosic fibers provide absorbent products with critical functionality that has, to date, not been duplicated by particulate superabsorbent polymers. Superabsorbent materials have been introduced in synthetic fiber form seeking to provide a material having the functionality of both fiber and superabsorbent polymer particle. However, these superabsorbent fibers are difficult to process compared to fluff pulp fibers and do not blend well with fluff pulp fibers. Furthermore, synthetic superabsorbent fibers are significantly more expensive than superabsorbent polymer particles and, as a result, have not competed effectively for high volume use in personal care absorbent products.

Cellulosic fibers have also been rendered highly absorptive by chemical modification to include ionic groups such as carboxylic acid, sulfonic acid, and quaternary ammonium groups that impart water swellability to the fiber. Although some of these modified cellulosic materials are soluble in water, some are water-insoluble. However, none of these highly absorptive modified cellulosic materials possess the structure of a pulp fiber, rather, these modified cellulosic materials are typically granular or have a regenerated fibril form.

A need exists for a highly absorbent material suitable for use in personal care absorbent products, the absorbent material having absorptive properties similar to synthetic, highly absorptive materials and at the same time offering the advantages of liquid wicking and distribution associated with fluff pulp fibers. Accordingly, there is a need for a fibrous superabsorbent that combines the advantageous liquid storage capacity of superabsorbent polymers and the advantageous liquid wicking of fluff pulp fibers. Ideally, the fibrous superabsorbent is economically viable for use in personal care absorbent products. The present invention seeks to fulfill these needs and provides further related advantages.

### SUMMARY OF THE INVENTION

In one aspect, the present invention provides a modified cellulosic fiber having superabsorbent properties. The modified fiber formed in accordance with the present invention has a fibrous structure substantially identical to the cellulosic fiber from which it is derived. More importantly, the modified fiber is a water-swellable, water-insoluble fiber that substantially retains its fibrous structure in its expanded, water-swelled state. The modified fiber is a sulfated and crosslinked cellulosic fiber having a liquid absorption capacity of at least about 4 g/g. In one embodiment, the modified fiber is an individual, crosslinked, sulfated cellulosic fiber. In another embodiment, the invention provides a rollgood that includes the modified fiber. In one embodiment, the rollgood includes other materials such as fibrous, binder, and absorbent materials. In another embodiment, the rollgood can be directly inserted as an absorbent core into an absorbent article.

In another aspect of the invention, methods for forming the modified cellulosic fiber are provided. In one embodiment of the method, a sulfated cellulosic fiber is crosslinked to an extent sufficient to render the fiber substantially insoluble in water. In another embodiment, a crosslinked cellulosic fiber is sulfated to provide the modified fiber. The sulfated cellulosic fiber can be prepared by reacting the fiber with sulfuric acid in an organic solvent.

In others aspects, the invention provides methods for using the modified fiber and absorbent composites and articles incorporating the modified fiber are also provided. In one embodiment, the invention provides an absorbent core having a liquid capacity of at least about 22 g/g. The absorbent core can be advantageously incorporated into an absorbent article.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-C are scanning electron microscope (SEM) photographs of representative fluff pulp fibers (bleached kraft southern pine fibers commercially available from Weyerhaeuser Company under the designation NB416) at 100× magnification (FIG. 1A), at 300× magnification (FIG. 1B), and at 1000× magnification (FIG. 1C);

FIGS. 2A-C are SEM photographs of representative modified fibers formed in accordance with the present invention from bleached kraft southern pine fibers (NB416) at 100× magnification (FIG. 2A), at 300× magnification (FIG. 2B), and at 1000× magnification (FIG. 2C);

FIGS. 3A and 3B are optical microscope photographs of representative modified fibers formed in accordance with the present invention, FIG. 3A illustrates modified fibers before contact with water and FIG. 3B illustrates modified fibers after contact with water; and

FIG. 4 is a graph illustrating the absorbent capacity for representative modified fibers formed in accordance with the present invention as a function of weight percent crosslinking applied to the fibers and sulfation reaction time (25 minutes, +; 35 minutes, ▪; 45 minutes, Δ).

### DETAILED DESCRIPTION OF THE PREFERRED EMBODIMENT

In one aspect, the present invention provides a modified cellulosic fiber having superabsorbent properties. The modified fiber formed in accordance with the present invention has a fibrous structure substantially identical to the cellulosic fiber from which it is derived. More importantly, the modified fiber is a water-swellable, water-insoluble fiber that substantially retains its fibrous structure in its expanded, water-swelled state. The cellulosic fiber formed in accordance with the invention is modified cellulosic fiber that has been sulfated and crosslinked. Water swellability is imparted to the cellulosic fiber through sulfation and intrafiber crosslinking renders the cellulosic fiber substantially insoluble in water. The modified cellulosic fiber has a degree of sulfate group substitution effective to provide advantageous water swellability. The modified cellulosic fiber is crosslinked to an extent sufficient to render the fiber water insoluble. The modified cellulosic fiber has a liquid absorption capacity that is increased compared to unmodified fluff pulp fibers. The modified fibers have a liquid absorption capacity of at least about 4 g/g.

Cellulosic fibers suitable for use in forming the modified fiber of the present invention are substantially water-insoluble and not highly water-swellable. After sulfation and crosslinking in accordance with the present invention, the resulting modified fiber has the desired absorbency characteristics, is water-swellable and water-insoluble, and substantially retains the fibrous structure of the cellulosic fiber from which it is derived.

The modified fiber of the invention has the structure of a pulp fiber including a cell wall structure. In one embodiment, the modified fiber has the structure of a wood pulp fiber. The modified fiber includes a lumen (i.e., central cavity) surrounded by a wall surface having four concentric layers. In addition to an outermost primary wall (commonly denoted P), the cell wall includes secondary walls (commonly denoted S1-S3). The secondary walls include an outer layer (S1) adjacent the primary wall, an inner layer (S3) adjacent the lumen, and a middle layer (S2) intermediate the outer and inner secondary layers. The modified fiber's structure also includes long bundles of cellulosic fibrillar structures, referred to as macrofibrils, fibrils, microfibrils, and elementary fibrils, having varying diameters. The diameter of fibrillar material depends on the extent of fiber processing.

Cellulose is a principal component of delignified cell walls. For example, the secondary cell wall can include unbranched cellulose chains having a degree of polymerization up to about 17,000. Accordingly, the modified fiber of the invention is primarily cellulosic in nature having cellulose as its principal chemical component. Cellulose can be considered to be a polymer containing repeating anhydroglucose units. The term “anhydroglucose” refers to the repeating unit in cellulose that is formed by loss of water from glucose on condensation to form the polymer. The degree of polymerization (DP) for a given cellulose molecule is the number of anhydroglucose repeating units in the molecule. The DP for a particular cellulose will depend on its source and the extent of polymer degradation on processing.

In addition to cellulose, the modified fiber can include hemicellulose and lignin. While cellulose is a linear polysaccharide formed from glucose, hemicellulose can be either an unbranched or branched polysaccharide that includes sugars other than glucose. Unlike cellulose and hemicellulose, which are carbohydrate polymers having repeating saccharide units, lignin is a highly branched, three-dimensional polymer composed of aromatic units. Lignin is amorphous in structure and not an integral part of the fiber's fibrillar system of carbohydrate polymers.

For native wood fibers, lignin content is greatest in the outer layers of the cell wall and decreases rapidly to the layer adjacent the lumen. In contrast, cellulose content is lowest in the primary wall and increases significantly toward the inner fiber regions. Hemicellulose content tends to increase gradually from the outer to the inner regions of the fiber. A description of the chemical composition and structure of wood fibers is provided in Pulp and Paper Manufacture, Volume I. The Pulping of Wood, Second Edition, R. G. MacDonald, Ed., MacGraw-Hill. 1969, pages 39-45.

The chemical composition of the modified fiber of the invention depends, in part, on the extent of processing of the cellulosic fiber from which the modified fiber is derived. In general, the modified fiber of the invention is derived from a fiber that has been subjected to a pulping process (i.e., a pulp fiber). Pulp fibers are produced by pulping processes that seek to separate cellulose from lignin and hemicellulose leaving the cellulose in fiber form. The amount of lignin and hemicellulose remaining in a pulp fiber after pulping will depend on the nature and extent of the pulping process.

Thus, the fiber of the invention is a modified pulp fiber that retains the basic chemical and structural characteristics of a pulp fiber. The modified fiber has a multiwalled macrostructure as described above and is composed of primarily of cellulose and can include some hemicellulose and lignin.

The modified fiber is substantially insoluble in water. As used herein, a material will be considered to be water-soluble when it substantially dissolves in excess water to form a solution, losing its fiber form and becoming essentially evenly disbursed throughout a water solution. A sufficiently sulfated cellulosic fiber that is free from a substantial degree of crosslinking will be water-soluble, whereas the modified cellulosic fiber of the invention, a sulfated and crosslinked fiber, is water-insoluble.

The modified fiber is a water-swellable, water-insoluble fiber. As used herein, the term “water-swellable, water-insoluble” refers to a material that, when exposed to an excess of an aqueous medium (e.g., bodily fluids such as urine or blood, water, synthetic urine, or 0.9 weight percent solution of sodium chloride in water), swells to an equilibrium volume but does not dissolve into solution. The water-swellable, water-insoluble modified cellulosic fibers of the invention retain their original fibrous structure, but in a highly expanded state, during liquid absorption and have sufficient structural integrity to resist flow and fusion with neighboring materials. A modified fiber of the invention is effectively crosslinked to be substantially insoluble in water while being capable of absorbing at least about 4 times its weight of a 0.9 weight percent solution of sodium chloride in water under an applied load of about 0.3 pound per square inch.

Cellulosic fibers are a starting material for preparing the superabsorbent cellulosic fiber product of the invention. Although available from other sources, suitable cellulosic fibers are derived primarily from wood pulp. Suitable wood pulp fibers for use with the invention can be obtained from well-known chemical processes such as the kraft and sulfite processes, with or without subsequent bleaching. Pulp fibers can also be processed by thermomechanical, chemithermomechanical methods, or combinations thereof. Caustic extractive pulp such as TRUCELL, commercially available from Weyerhaeuser Company, is also a suitable wood pulp fiber. The preferred pulp fiber is produced by chemical methods. Ground wood fibers, recycled or secondary wood pulp fibers, and bleached and unbleached wood pulp fibers can be used. Softwoods and hardwoods can be used. Details of the selection of wood pulp fibers are well-known to those skilled in the art. These fibers are commercially available from a number of companies, including Weyerhaeuser Company, the assignee of the present invention. For example, suitable cellulosic fibers produced from southern pine that are usable with the present invention are available from Weyerhaeuser Company under the designations CF416, NF405, PL416, FR516, and NB416. In one embodiment, the cellulosic fiber useful in making the modified fiber of the invention is a southern pine fiber commercially available from Weyerhaeuser Company under the designation NB416. In other embodiments, the cellulosic fiber can be selected from among a northern softwood fiber, a eucalyptus fiber, a rye grass fiber, and a cotton fiber.

Cellulosic fibers having a wide range of degree of polymerization are suitable for forming the modified cellulosic fiber of the invention. In one embodiment, the cellulosic fiber has a relatively high degree of polymerization, greater than about 1000, and in another embodiment, about 1500.

In one embodiment, the modified fiber has an average length greater than about 1.0 mm. Consequently, the modified fiber is suitably prepared from fibers having lengths greater than about 1.0 mm. Fibers having lengths suitable for preparing the modified fiber include southern pine, northern softwood, and eucalyptus fibers, the average length of which is about 2.8 mm, about 2.0 mm, and about 1.5 mm, respectively. Fibers with average lengths less than about 1.0 mm have relatively poorer wicking properties and provide composites having diminished pad integrity.

The modified cellulosic fiber of the invention is a sulfated cellulosic fiber. As used herein, “sulfated cellulosic fiber” refers to a cellulosic fiber that has been sulfated by reaction of a cellulosic fiber with a sulfating agent. It will be appreciated that the term “sulfated cellulosic fiber” includes free acid and salt forms of the sulfated fiber. Suitable metal salts include sodium, potassium, and lithium salt, among others. A sulfated cellulosic fiber can be produced by reacting a sulfating agent with a hydroxyl group of the cellulosic fiber to provide a cellulose sulfate ester (i.e., a carbon-to-oxygen-to-sulfur ester). The sulfated cellulosic fiber formed in accordance with the present invention differs from other sulfur-containing cellulosic compounds in which the sulfate sulfur atom is attached directly to a carbon atom on the cellulose chain as, for example, in the case of sulfonated cellulose; or cellulosic compounds in which the sulfate sulfur atom is attached indirectly to a carbon atom on the cellulose chain as, for example, in the case of cellulose alkyl sulfonates.

The modified cellulosic fiber of the invention can be characterized as having an average degree of sulfate group substitution of from about 0.1 to about 2.0. In one embodiment, the modified cellulosic fiber has an average degree of sulfate group substitution of from about 0.2 to about 1.0. In another embodiment, the modified cellulosic fiber has an average degree of sulfate group substitution of from about 0.3 to about 0.5. As used herein, the “average degree of sulfate group substitution” refers to the average number of moles of sulfate groups per mole of glucose unit in the modified fiber. It will be appreciated that the fibers formed in accordance with the present invention include a distribution of sulfate modified fibers having an average degree of sulfate substitution as noted above.

A representative method for preparing sulfated fibers is described in Example 1.

The modified cellulosic fiber of the invention is an intrafiber crosslinked cellulosic fiber. Crosslinked cellulosic fibers and methods for their preparation are disclosed in U.S. Pat. Nos. 5,437,418 and 5,225,047 issued to Graef et al., expressly incorporated herein by reference.

Crosslinked fibers can be prepared by treating fibers with a crosslinking agent. Suitable crosslinking agents useful in producing the modified cellulosic fiber are generally soluble in water and/or alcohol. Suitable cellulosic fiber crosslinking agents include aldehyde, dialdehyde, and related derivatives (e.g., formaldehyde, glyoxal, glutaraldehyde, glyceraldehyde), and urea-based formaldehyde addition products (e.g., N-methylol compounds). See, for example, U.S. Pat. Nos. 3,224,926; 3,241,533; 3,932,209; 4,035,147; 3,756,913; 4,689,118; 4,822,453; U.S. Pat. No. 3,440,135, issued to Chung; U.S. Pat. No. 4,935,022, issued to Lash et al.; U.S. Pat. No. 4,889,595, issued to Herron et al.; U.S. Pat. No. 3,819,470, issued to Shaw et al.; U.S. Pat. No. 3,658,613, issued to Steiger et al.; and U.S. Pat. No. 4,853,086, issued to Graef et al., all of which are expressly incorporated herein by reference in their entirety. Cellulosic fibers can also be crosslinked by carboxylic acid crosslinking agents including polycarboxylic acids. U.S. Pat. Nos. 5,137,537; 5,183,707; and 5,190,563, describe the use of C2-C9 polycarboxylic acids that contain at least three carboxyl groups (e.g., citric acid and oxydisuccinic acid) as crosslinking agents.

Suitable urea-based crosslinking agents include methylolated ureas, methylolated cyclic ureas, methylolated lower alkyl substituted cyclic ureas, methylolated dihydroxy cyclic ureas, dihydroxy cyclic ureas, and lower alkyl substituted cyclic ureas. Specific preferred urea-based crosslinking agents include dimethylol urea (DMU, bis[N-hydroxymethyl]urea), dimethylolethylene urea (DMEU, 1,3-dihydroxymethyl-2-imidazolidinone), dimethyloldihydroxyethylene urea (DMDHEU, 1,3-dihydroxymethyl-4,5-dihydroxy-2-imidazolidinone), dimethylolpropylene urea (DMPU), dimethylolhydantoin (DMH), dimethyldihydroxy urea (DMDHU), dihydroxyethylene urea (DHEU, 4,5-dihydroxy-2-imidazolidinone), and dimethyldihydroxyethylene urea (DMeDHEU, 4,5-dihydroxy-1,3-dimethyl-2-imidazolidinone).

Suitable polycarboxylic acid crosslinking agents include citric acid, tartaric acid, malic acid, succinic acid, glutaric acid, citraconic acid, itaconic acid, tartrate monosuccinic acid, maleic acid, 1,2,3-propane tricarboxylic acid, 1,2,3,4-butanetetracarboxylic acid, all-cis-cyclopentane tetracarboxylic acid, tetrahydrofuran tetracarboxylic acid, 1,2,4,5-benzenetetracarboxylic acid, and benzenehexacarboxylic acid. Other polycarboxylic acids crosslinking agents include polymeric polycarboxylic acids such as poly(acrylic acid), poly(methacrylic acid), poly(maleic acid), poly(methylvinylether-co-maleate) copolymer, poly(methylvinylether-co-itaconate) copolymer, copolymers of acrylic acid, and copolymers of maleic acid. The use of polymeric polycarboxylic acid crosslinking agents such as polyacrylic acid polymers, polymaleic acid polymers, copolymers of acrylic acid, and copolymers of maleic acid is described in U.S. Pat. No. 5,998,511, assigned to Weyerhaeuser Company and expressly incorporated herein by reference in its entirety.

Other suitable crosslinking agents include diepoxides such as, for example, vinylcyclohexene dioxide, butadiene dioxide, and diglycidyl ether; sulfones such as, for example, divinyl sulfone, bis(2-hydroxyethyl)sulfone, bis(2-chloroethyl)sulfone, and disodium tris(β-sulfatoethyl)sulfonium inner salt; and diisocyanates.

Mixtures and/or blends of crosslinking agents can also be used.

The crosslinking agent can include a catalyst to accelerate the bonding reaction between the crosslinking agent and cellulosic fiber. Suitable catalysts include acidic salts, such as ammonium chloride, ammonium sulfate, aluminum chloride, magnesium chloride, and alkali metal salts of phosphorous-containing acids.

The modified cellulosic fiber of the invention is a crosslinked cellulosic fiber. The amount of crosslinking agent applied to the fiber is suitably the amount necessary to render the modified fiber substantially insoluble in water. The amount of crosslinking agent applied to the cellulosic fiber will depend on the particular crosslinking agent and is suitably in the range of from about 0.01 to about 8.0 percent by weight based on the total weight of cellulosic fiber. In one embodiment, the amount of crosslinking agent applied to the fibers is in the range from about 0.20 to about 5.0 percent by weight based on the total weight of fibers.

In one embodiment, the crosslinking agent can be applied to the cellulosic fibers as an aqueous alcoholic solution. Water is present in the solution in an amount sufficient swell the fiber to an extent to allow for crosslinking within the fiber's cell wall. However, the solution does not include enough water to dissolve the fiber. Suitable alcohols include those alcohols in which the crosslinking agent is soluble and the fiber to be crosslinked (i.e., unmodified or sulfated cellulosic fiber) is not. Representative alcohols include alcohols that include from 1 to 5 carbon atoms, for example, methanol, ethanol, n-propanol, isopropanol, n-butanol, isobutanol, s-butanol, and pentanols. In another embodiment, the crosslinking agent can be applied to the fibers as an ether solution (e.g., diethyl ether).

It will be appreciated that due to its fiber structure, the modified fiber of the invention can have a distribution of sulfate and/or crosslinking groups along the fiber's length and through the fiber's cell wall. Generally, there can be greater sulfation and/or crosslinking on or near the fiber surface than at or near the fiber core. Surface crosslinking may be advantageous to improve modified fiber dryness and provide a better balance of total absorbent capacity and surface dryness. Fiber swelling and soak time can also effect the sulfation and crosslinking gradients. Such gradients may be due to the fiber structure and can be adjusted and optimized through control of sulfation and/or crosslinking reaction conditions.

A representative method for crosslinking sulfated fibers is described in Example 2.

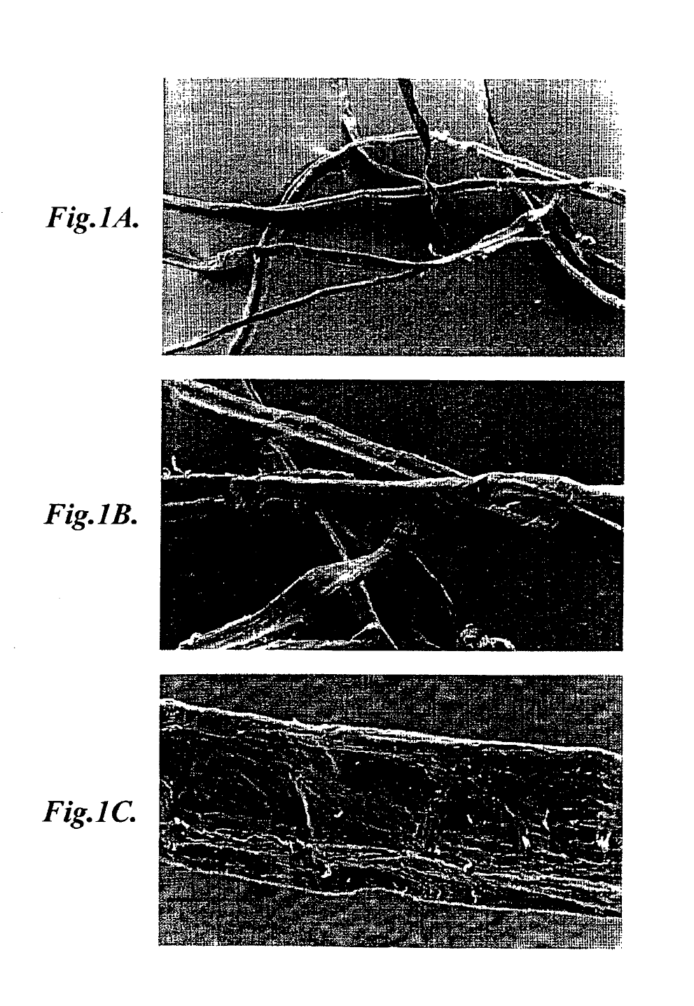

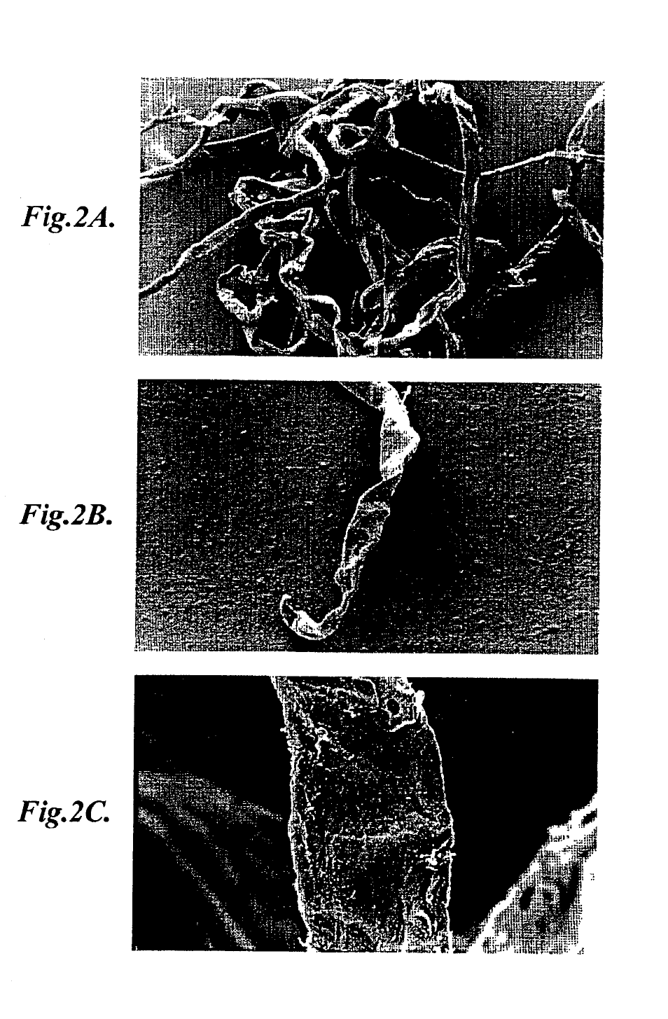

Scanning electron microscope (SEM) photographs of bleached kraft southern pine fibers (NB416) at 100×, 300×, and 1000× magnification are illustrated in FIGS. 1A-C, respectively. SEM photographs of representative modified fibers formed from NB416 fibers in accordance with the invention at 100×, 300×, and 1000× magnification are illustrated in FIGS. 2A-C, respectively. Referring to FIGS. 1A-C and 2A-C, the modified fibers are ribbon-like and are twisted and curled, and have a structure substantially identical to the fiber from which they are derived.

The modified fiber of the invention has a liquid absorbent capacity of at least about 4 g/g as measured by the centrifuge capacity test described in Example 3. In one embodiment, the modified fiber has a capacity of at least about 10 g/g. In another embodiment, the fiber has a capacity of at least about 15 g/g, and in a further embodiment, the fiber has a capacity of at least about 20 g/g. The absorbent capacity of representative modified fibers formed in accordance with the present invention is described in Example 3.

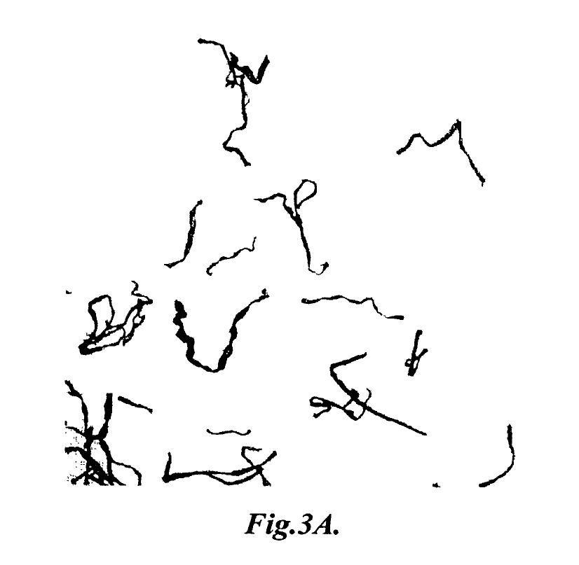

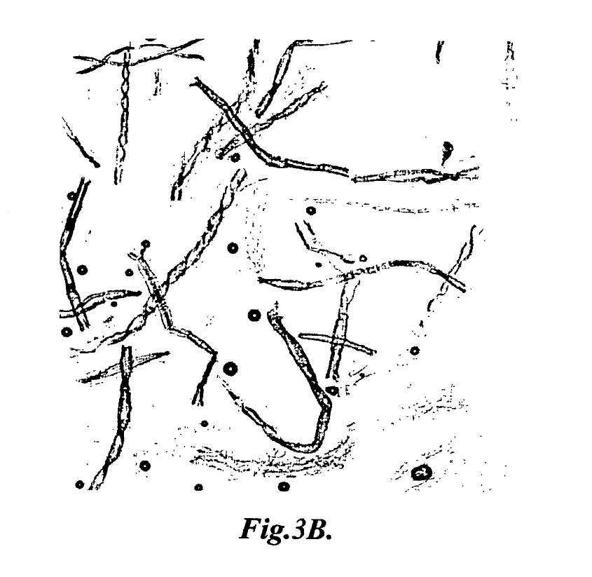

As noted above, the modified fiber retains the structure of a fiber. FIGS. 3A and 3B are optical microscope photographs of representative modified fibers formed in accordance with the invention before and after contact with water. FIG. 3A shows representative modified fibers that have not been contacted with water. Referring to FIG. 3A, these fibers are ribbon-like and are twisted and curled. FIG. 3B shows representative modified fibers that have been contacted with water. Referring to FIG. 3B, these swelled fibers have retained their fiber structure and have expanded diameters that are from about 3 to about 6 times their original diameter.

In another aspect of the invention, methods for making a cellulosic fiber having superabsorbent properties are provided. In the methods, cellulosic fibers are sulfated and crosslinked to provide superabsorbent fibers. In one embodiment, cellulosic fibers are sulfated and then crosslinked. In this method, sulfated cellulosic fibers are treated with an amount of crosslinking agent sufficient to render the resulting modified cellulosic fibers substantially insoluble in water. In another embodiment, cellulosic fibers are crosslinked then sulfated. In this method, crosslinked cellulosic fibers are sulfated to render the resulting modified cellulosic fibers highly water absorptive. The modified cellulosic fiber formed by either method is highly water absorptive, water-swellable, water-insoluble, and retains the fibrous structure of the fibers from which it is derived.

The modified fiber of the invention is a sulfated cellulosic fiber. Sulfated cellulosic fibers can be made by reacting cellulosic fibers (e.g., cellulosic fibers that are crosslinked or noncrosslinked) with a sulfating agent. Suitable sulfating agents include concentrated sulfuric acid (95-98%), fuming sulfuric acid (oleum), sulfur trioxide and related complexes including sulfur trioxide/dimethylformamide and sulfur trioxide/pyridine complexes, and chlorosulfonic acid, among others. In one embodiment, the sulfating agent is concentrated sulfuric acid.

The sulfating agent is preferably applied to the fibers as a solution in an organic solvent. Suitable organic solvents include alcohols, pyridine, dimethylformamide, acetic acid including glacial acetic acid, and dioxane. In one embodiment, the organic solvent is an alcohol having up to about 6 carbon atoms. Suitable alcohols include methanol, ethanol, n-propanol, isopropanol, n-butanol, isobutanol, s-butanol, pentanols, and hexanols. In one embodiment, the alcohol is selected from among isopropanol and isobutanol.

The molar ratio of sulfuric acid to alcohol in the solution can be varied from about 1:1 to about 4:1. In one embodiment, the molar ratio of sulfuric acid to alcohol is about 2.4:1, for example, an 80:20 (weight/weight) solution of sulfuric acid in isopropanol. The weight ratio of sulfuric acid to cellulosic fibers in the sulfation reaction can be varied from about 5:1 to about 30:1. At low sulfuric acid ratios the reaction is slow and incomplete and at high sulfuric acid ratios significant cellulose polymer degradation can occur. In one embodiment, the weight ratio of sulfuric acid to pulp fiber is from about 10:1 to about 25:1. In another embodiment, the weight ratio of sulfuric acid to pulp fiber is about 24:1.

Highly acidic aqueous environments readily degrade cellulose fibers. It has been reported that concentrated sulfuric acid cannot be used to prepare sulfated cellulose because treating cellulose with sulfuric acid results in a soluble product formed from acid hydrolysis of the cellulose backbone by the sulfuric acid. See, WO 96/15137. However, a water-soluble cellulose sulfate has been reportedly prepared from an activated cellulose (20 to 30% water) by direct action of aqueous sulfuric acid or sulfuric acid dissolved in a volatile organic solvent such as toluene, carbon tetrachloride, or a lower alkanol. “Cellulose Chemistry and Its Applications”, Ed. T. P. Nevell and S. H. Zeronian, Halstead Press. John Wiley and Sons, 1985. page 350.

Despite the well-known degradation of cellulose in aqueous acidic solutions, the present invention provides methods for making sulfated cellulose fibers without significant cellulose hydrolysis. In the methods of the invention, cellulose fiber degradation (i.e., degree of polymerization reduction) is substantially avoided by treating cellulose fibers with a sulfating agent in a nonaqueous environment and/or at low temperature (e.g., at or below about 4° C.). To further protect against fiber degradation (e.g., hydrolysis), a dehydrating agent to absorb water, including water formed during the sulfation reaction, can be added to the sulfating reaction mixture. Suitable dehydrating agents include, for example, sulfur trioxide, magnesium sulfate, acetic anhydride, and molecular sieves. In one embodiment, cellulosic fibers are reacted with the sulfating agent at a temperature of about 4° C. and both the cellulosic fibers and the sulfating agent are cooled to about 4° C. prior to reaction. In another embodiment, cellulosic fibers, including cooled fibers, are reacted with the sulfating agent in the presence of a dehydrating agent.

Depending upon the extent of sulfation desired, the fibers and sulfating agent are reacted for a period of time of from about 10 to about 60 minutes. Following this reaction period and prior to neutralizing the resulting sulfated fibers, the sulfated fibers are separated from excess sulfating agent. In one embodiment, the sulfated fibers are washed with an alcohol prior to neutralization.

Prior to crosslinking the sulfated cellulosic fibers to provide the modified fibers of the invention, the fibers can be at least partially neutralized with a neutralizing agent. The neutralizing agent is suitably soluble in the sulfation solvent. In one embodiment, the neutralizing agent is a base such as, for example, an alkaline base (e.g., lithium, potassium, sodium or calcium hydroxide; lithium, potassium, or sodium acetate). Alternatively, the neutralizing agent can include a multivalent metal salt. Suitable metal salts include cerium, magnesium, calcium, zirconium, and aluminum salts such as ammonium cerium nitrate, magnesium sulfate, magnesium chloride, calcium chloride, zirconium chloride, aluminum chloride, and aluminum sulfate, among others. The use of multivalent metal salts as neutralizing agents also offers the advantage of intrafiber crosslinking. Thus, through the use of a multivalent metal salt, the sulfated cellulosic fiber can be partially neutralized and partially crosslinked. Fibers so treated can be further crosslinked with other crosslinking agents including those described above.

The extent of fiber sulfation is dependent on a number of reaction conditions including reaction time. For example, in a series of representative sulfation reactions, a 25 minute reaction time provided a fiber that included about 3.8 percent by weight sulfur; a 35 minute reaction time provided a fiber that included about 4.9 percent by weight sulfur; and a 45 minute reaction time provided a fiber that included about 6.4 percent by weight sulfur. However, in these experiments, the extended sulfation reaction time had an adverse effect on fiber length (i.e., cellulose hydrolysis occurred under the prolonged reaction conditions). In viscosity experiments, the sulfated fibers produced by the 25 and 35 minute reaction conditions provide cellulose solutions classified as having a Gardner-Holt bubble tube H viscosity (i.e., about 200 Centistokes), while the sulfated fibers produced by the 45 minute reaction provided cellulose solutions classified as having C viscosity (i.e., about 85 Centistokes). The results indicate that at extended reaction times, significant fiber degradation can occur. The absorbent capacity of modified fibers prepared from these sulfated fibers is described in Example 3.

A representative method for preparing sulfated fibers is described in Example 1.

The at least partially neutralized sulfated cellulosic fibers can then be crosslinked by applying a crosslinking agent to the fibers. In one embodiment, the crosslinking agent is applied to the fibers as an aqueous alcoholic solution. In general, the crosslinking agent solution includes water sufficient to swell but not dissolve the fibers. Above about 95 percent by weight alcohol, the crosslinking agent does not penetrate the fiber cell wall sufficiently and the result is a crosslinked fiber having nonuniform crosslinking and low absorbent capacity. Suitably, the aqueous alcoholic solution includes from about 10 to about 50 percent by weight water and from about 50 to about 90 percent by weight alcohol. In one embodiment, the crosslinking agent solution is an aqueous ethanol solution (88 percent by weight ethanol).

After the fibers have been treated with the crosslinking agent, the crosslinking agent is cured by, for example, heating the treated fibers, to provide intrafiber crosslinked fibers.

A representative method for crosslinking sulfated fibers is described in Example 2. The method of Example 2 describes crosslinking sulfated fibers that have been isolated and dried. Alternatively, sulfated fibers formed as described above and in Example 1 may be directly crosslinked, after neutralization, without drying the fibers.

Thus, in one embodiment, the present invention provides a method for making cellulosic fibers having superabsorbent properties that includes the step of reacting cellulosic fibers with a sulfating agent, at least partially neutralizing the sulfated fibers to provide fibers suitable for crosslinking, applying a crosslinking agent to the sulfated fibers, and then curing the crosslinking agent to provide the modified fibers.

It has been discovered that the nature of the modified fiber of the present invention can be varied and controlled by the amount of water present in the crosslinking reaction. For example, when it is desirable to produce the modified fiber in individual fiber form, relatively less water is used in the crosslinking reaction. Conversely, when it is desired that the modified fiber be produced as a sheet or web (e.g., rollgood), the crosslinking reaction includes a relatively greater amount of water. It has been found that water present during the crosslinking reaction effects bonding between the individual, modified fibers. When the water content is sufficiently high in the crosslinking reaction, interfiber bonding can occur to provide a structure having sufficient strength and integrity to provide a fibrous web or sheet of the modified fiber suitable for the formation of a rollgood. Where it is desirable to form the modified fiber in individual form, the modified fiber can be baled for shipping and subsequent processing.

Some interfiber bonding and loss of individual fiber structure occurs when more than about 50 percent by weight water is present in the crosslinking reaction. Between from about 50 and about 90 percent by weight alcohol, interfiber bonding occurs without the loss of individual fiber structure.

The method described above can further include other steps to optimize the production of the modified fibers of the invention. To further assist in preventing fiber hydrolysis during sulfation, the cellulosic fibers can be dried prior to the sulfation reaction. The fibers can be dried by any one of a number of drying methods including heating and chemical methods. For example, the fibers can be dried by heating in a drying oven; solvent exchange with a suitable solvent; solvent exchange with a suitable solvent followed by heating; or treatment with a dehydrating agent such sulfur trioxide or acetic anhydride. Alternatively, a never-dried fiber can be dried by solvent exchange using a suitable solvent.

For effective sulfation, cellulosic fibers, including dried fibers, can be swelled prior to sulfation using a swelling agent. Suitable swelling agents include, for example, water, glacial acetic acid, acetic anhydride, zinc chloride, sulfuric acid, sulfur trioxide, and ammonia. The fibers can be swelled by mixing the fibers with the swelling agent followed by removing excess swelling agent prior to reacting the fibers with the sulfating agent.

Thus, in another embodiment, the present invention provides a method for making cellulosic fibers having superabsorbent properties that includes the steps of swelling cellulosic fibers, including dry fibers, with a swelling agent; separating excess swelling agent from the swelled fibers; reacting the swelled fibers with a sulfating agent; separating excess sulfating agent from the fibers; at least partially neutralizing the sulfated fibers to provide fibers suitable for crosslinking; applying a crosslinking agent to the sulfated fibers; and then curing the crosslinking agent to provide intrafiber crosslinked, sulfated cellulosic fibers.

In another embodiment, the modified cellulosic fibers of the invention can be formed by crosslinking then sulfating the cellulosic fibers. In the method, the modified fibers can be prepared by applying a crosslinking agent to cellulosic fibers; curing the crosslinking agent to provide crosslinked fibers; reacting the crosslinked cellulosic fibers with a sulfating agent; at least partially neutralizing the sulfated, crosslinked fibers; and then drying the sulfated, crosslinked cellulosic fibers.

The modified fiber of the invention is formed by methods that do not include dissolving the fiber in solution. In this way, the modified fiber retains the structure of the fiber from which it is derived. The structure of the modified fiber of the invention is in contrast to other fibrous materials that lack fiber structure and that are prepared by regeneration from solutions (i.e., formed, for example, by precipitation, from solutions containing dissolved cellulosic materials).

The modified fiber formed in accordance with the present invention has superabsorbent properties while, at the same time, has the structure of the cellulosic pulp fiber from which it is derived. As noted above, the modified fiber of the invention can be produced as an individual fiber or as sheet or web (e.g., rollgood) of fibers. The nature of the modified fiber produced depends on the use for which the fiber is ultimately intended.

The modified fibers can be incorporated into a personal care absorbent product. The modified fibers can be formed into a composite for incorporation into a personal care absorbent product. Composites can be formed from the modified fibers alone or by combining the modified fibers with other materials, including fibrous materials, binder materials, other absorbent materials, and other materials commonly employed in personal care absorbent products. Suitable fibrous materials include synthetic fibers, such as polyester, polypropylene, and bicomponent binding fibers; and cellulosic fibers, such as fluff pulp fibers, crosslinked cellulosic fibers, cotton fibers, and CTMP fibers. Suitable absorbent materials include natural absorbents, such as sphagnum moss, and synthetic superabsorbents, such as polyacrylates (e.g., SAPs).

In one embodiment, the modified fiber is further treated with a compatible material to provide a coated modified fiber. The modified fiber can be coated with a variety of materials including those noted above as well as binders, pH control agents, and odor reducing agents, among others.

Webs that include the modified fibers can be prepared in any one of a variety of methods known in the web-forming art. The methods include airlaid and wet forming methods. As noted above, wet-formed webs that include the modified fibers can be formed by, for example, adding water in an amount sufficient to bond the crosslinked sulfated fibers to an extent sufficient to provide a web with structural integrity. Other materials, such as fibrous and absorbent materials, can also be included in these webs.

In some instances, when intended for use in a personal care absorbent product, the rollgood form of the modified fiber is desired. One advantage of the modified fiber in rollgood form is that it can be directly incorporated as received by a diaper manufacturer by cutting the rollgood into the desired shape and size, and inserting the shaped and sized web into an absorbent article. In this way, the modified fiber in rollgood form can be directly utilized in a diaper manufacturing line. The rollgood containing the modified fiber can also include any one or more of a variety of other useful materials such as those identified above.

Absorbent composites derived from or that include the modified fibers of the invention can be advantageously incorporated into a variety of absorbent articles such as diapers including disposable diapers and training pants; feminine care products including sanitary napkins, and pant liners; adult incontinence products; toweling; surgical and dental sponges; bandages; food tray pads; and the like. Thus, in another aspect, the present invention provides absorbent composites and absorbent articles that include the modified fiber.

As noted above, the modified fiber of the invention has a fiber structure that, like other pulp fibers, provides for liquid wicking. Like superabsorbent materials, the modified fiber has a high liquid absorbent capacity. Accordingly, the modified fiber can be useful in absorbent products such as, for example, an infant diaper, where liquid wicking and liquid storage are required. Because of its unique, liquid wicking and capacity properties, the modified fiber can be formed into a composite and utilized as a storage core in a diaper. Such a core may only include the modified fiber. For a modified fiber having an absorbent capacity of at least about 22 g/g, the resulting core has an absorbent capacity of at least about 22 g/g. Conventional, commercial diaper storage cores typically include two components: (1) fluff pulp fibers to wick liquid, and (2) superabsorbent material to store acquired liquid. The core typically consists of minimally about 25 percent by weight fluff pulp fibers and maximally about 75 percent by weight superabsorbent material. Superabsorbent materials generally have an absorbent capacity of about 28 g/g and fluff pulp fibers generally have an absorbent capacity of about 2 g/g. Therefore, such a core has a capacity of about 22 g/g. Cores prepared from a modified fiber having a capacity of at least about 22 g/g can exceed the performance characteristics of conventional absorbent composites. Thus, the modified fibers of the invention provide advantages related to the manufacture of absorbent cores.

The following examples are provided for the purposes of illustrating, not limiting, the present invention.

### EXAMPLES

### Example 1

The Preparation of Sulfated Cellulosic Fibers

In this example, a representative method for forming sulfated cellulosic fibers is described.

Prior to sulfation, the pulp was activated with acetic acid. Ten grams of fiberized bleached kraft southern yellow pine fluff pulp (NB416, Weyerhaeuser Company, Federal Way, Wash.) that had been oven dried at 105° C. was disbursed in 600 mL of glacial acetic acid. The pulp/acid slurry was then placed in a vacuum chamber and the air was evacuated. The slurry was allowed to stand under vacuum for 30 minutes after which time the chamber was repressurized to atmospheric pressure. The slurry was then allowed to stand at ambient conditions for 45 minutes before being resubjected to a vacuum for an additional 30 minutes. After the second application of a vacuum the slurry was again allowed to stand for 45 minutes at atmospheric pressure. The slurry was then poured into a Buchner funnel where the pulp was collected and pressed until the weight of the residual acetic acid was equal to twice the weight of the oven dry pulp (i.e., total weight of the collected pulp was 30 g.) The collected pulp was placed inside a plastic bag and cooled to −10° C. in a freezer.

The sulfation liquor was prepared by mixing 240 g concentrated sulfuric acid with 60 g isopropanol and 0.226 g magnesium sulfate. The liquor was prepared by pouring isopropanol into a beaker that was maintained at 4° C. in an ice bath. Magnesium sulfate was then added to the isopropanol and the mixture chilled to 4° C. Sulfuric acid was weighed into a beaker and separately chilled to 9° C. before being slowly mixed into the isopropanol and magnesium sulfate mixture. The resulting sulfating liquor was then allowed to cool to 4° C.

The cooled acetic acid activated pulp (−10° C.) was stirred into the cooled sulfation liquor (4° C.). The resulting slurry of pulp and sulfation liquor was allowed to react for 35 minutes with constant stirring. After 35 minutes the pulp/sulfation liquor slurry was poured into a Buchner funnel and the sulfated pulp was collected and washed over a vacuum with cooled isopropanol (−10° C.). The collected pulp was then slurried with cooled isopropanol (−10° C.) in a Waring blender and poured back into the Buchner funnel where the pulp was again washed with cooled isopropanol (−10° C.).

The nature and quality of the modified fiber formed in accordance with the invention can depend on the washing step. First, the acid is preferably washed from the pulp as quickly as possible to prevent continued and/or accelerated cellulose degradation. Second, the cool temperature of the pulp is preferably maintained to prevent cellulose degradation. Third, the acid is preferably washed from the pulp as thoroughly as possible before neutralization to prevent the formation of difficult to remove inorganic salts during the neutralization step. These salts can adversely impact modified fiber absorbency.)

The washed sulfated pulp was next slurried in cooled isopropanol (−10° C.) and an ethanolic sodium hydroxide solution was added dropwise until the slurry was neutralized. The slurry was then poured into a Buchner funnel where the neutralized sulfated pulp was washed with room temperature isopropanol. The neutralized sulfated pulp was then agitated to remove any inorganic salts that may have been crusted on the fiber surfaces after which the neutralized sulfated pulp was again washed with isopropanol in a Buchner funnel. Finally the collected sulfated pulp was allowed to air dry.

### Example 2

The Preparation of Representative Crosslinked, Sulfated Cellulosic Fibers

In this example, a representative method for forming crosslinked, sulfated cellulosic fibers is described. Sulfated cellulosic fibers prepared as described in Example 1 were crosslinked with a representative crosslinking agent.

A catalyzed urea-formaldehyde system was used to crosslink the sulfated cellulosic fibers. The catalyst included magnesium chloride and the sodium salt of dodecylbenzenesulfonic acid dissolved in 88% ethanol/water. In addition to its primary function, the catalyst solution served as a diluent for the crosslinking agent. The crosslinking agent was obtained by dissolving urea in 37 percent (w/w) aqueous formaldehyde. The crosslinking agent was combined with the catalyst solution and applied to the sulfated fibers. The treated fibers were then cured by placing in a 105° C. oven for 60 minutes.

In the experiment, varying amounts of crosslinking agents were applied to the fibers. The amount of crosslinking agent used ranged from 1-11 percent of the weight of the sulfated fibers and the amount of catalytic diluent used was 250 percent of the weight of the sulfated fibers. The materials and their amounts used in preparing the catalytic diluent and crosslinking agent solutions are shown in Table 1 below.

TABLE 1
Composition of Catalytic Diluent and Crosslinking Agent Solution.

| | Parts |
|---|---|
| Catalytic Diluent | |
| Denatured ethanol | 44 |
| Deionized water | 6 |
| Magnesium chloride heptahydrate | 0.214 |
| Dodecylbenzenesulfonic acid, sodium salt | 0.4 |
| Crosslinking Agent Solution | |
| Urea | 15 |
| 37% (w/w) Formaldehyde | 41 |

### Example 3

The Performance Characteristics of Representative Crosslinked, Sulfated Cellulosic Fibers

In this example the performance characteristics of representative crosslinked, sulfated cellulosic fibers formed in accordance with the present invention is described. Representative modified fibers prepared as described in Examples 1 and 2 above, with varying levels of crosslinking agent applied to the fibers were evaluated for absorbent capacity by the total absorptive capacity/tea bag gel volume test described below. Modified fiber absorbent capacity as a function of crosslinking agent applied to the fiber is summarized in Table 2 below.

The preparation of materials, test procedure, and calculations to determine absorbent capacity were as follows.

Preparation of Materials:

1) Tea bag preparation: unroll tea bag material (Dexter #1234T heat-sealable tea bag material) and cut cross ways into 6 cm pieces. Fold lengthwise, outside-to-outside. Heatseal edges ⅛ inch with an iron (high setting), leave top end open. Trim excess from top edge to form a 6 cm×6 cm bag. Prepare 3 tea bags.

2) Label edge with sample identification.

3) Preweigh tea bag and record weight (to nearest 0.001 g).

4) Weigh 0.200 g sample (nearest 0.001 g) on tared glassine and record weight.

5) Fill tea bags with modified fiber sample.

6) Seal top edge of tea bag ⅛ inch with the iron.

7) Weigh and record total weight of tea bag filled with modified fiber sample. Store in sealed plastic bag until ready to test.

Test Procedure:

1) Fill container to a depth of at least 2 inch with 1 percent by weight saline solution.

2) Hold tea bag horizontally and distribute modified fiber sample evenly throughout tea bag.

3) Lay tea bag on the liquid surface of the saline solution (begin timing) and allow tea bag to wet-out before submerging the tea bag (about 10 sec.).

4) Soak tea bag for 30 minutes.

5) Remove tea bag from the saline solution with tweezers and clip to a drip rack.

6) Allow tea bag to hang for 3 minutes.

7) Carefully remove tea bag from clip and lightly touch saturated corner of tea bag on blotter to remove excess fluid. Weigh tea bag and record weight (i.e., drip weight).

8) Place tea bag on wall of centrifuge by pressing top edge against the wall. Balance centrifuge by placing the tea bags around the centrifuge's circumference.

9) Centrifuge at 2800 rpm for 75 seconds.

10) Remove tea bag from centrifuge, weigh and record tea bag centrifuged weight.

Absorbent Centrifuge Capacity Calculation:

(Net wet weight sample−Net dry weight sample)/Net dry weight sample=g/g capacity

Net wet weight is the centrifuge weight less the dry weight of the tea bag and fiber sample. Net dry weight is the dry weight of the fiber sample.

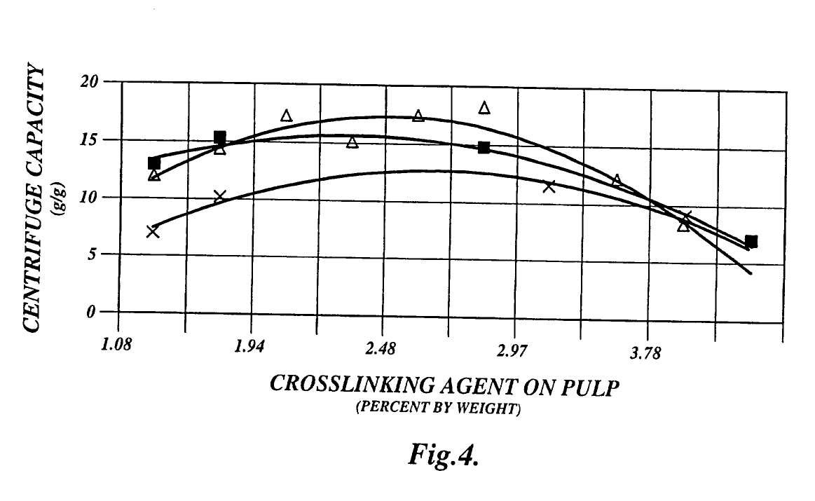

The absorbent capacity (g/g), determined as described above, as a function of sulfation reaction time and crosslinking agent applied to the fiber for representative modified fibers is summarized in Table 2 below and illustrated graphically in FIG. 4.

TABLE 2
Modified Fiber Absorbent Capacity: Crosslinking Level and
Sulfation Reaction Time Effect.

| Crosslinking | Centrifuge Capacity (g/g) | | |
|---|---|---|---|
| level (percent | 25 | 35 | 45 |
| by weight) | minute sulfation | minute sulfation | minute sulfation |
| 1.08 | 13.0 | 12.1 | 7.0 |
| 1.62 | 15.3 | 14.6 | 10.1 |
| 1.94 | | 17.2 | |
| 2.27 | | 15.1 | |
| 2.48 | | 17.3 | |
| 2.27 | 14.7 | 18.0 | |
| 2.97 | | | 11.3 |
| 3.24 | | 11.9 | |
| 3.78 | 8.1 | 7.9 | 8.6 |
| 4.00 | 6.6 | | |

As shown in Table 2 and FIG. 4, to a point, absorbent capacity increases with increasing sulfation. However, at the point where sulfation results in fiber degradation, absorbent capacity decreases. The results also demonstrate that absorbent capacity also increases with increasing crosslinking to a point. At higher levels of crosslinking, absorbent capacity decreases.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A modified cellulosic fiber, comprising a sulfated cellulosic fiber crosslinked to an extent to render the fiber substantially insoluble in water.

2. The fiber of claim 1 having a liquid absorption capacity of at least about 4 g/g.

3. The fiber of claim 1, wherein the average degree of sulfate substitution is from about 0.1 to about 2.0.

4. The fiber of claim 1, wherein the average degree of sulfate substitution is from about 0.2 to about 1.0.

5. The fiber of claim 1, wherein the average degree of sulfate substitution is from about 0.3 to about 0.5.

6. The fiber of claim 1, wherein the cellulosic fiber is a wood pulp fiber.

7. Individual, water-swellable, water-insoluble, intrafiber crosslinked, sulfated cellulosic fibers.

8. The fibers of claim 7 having a liquid absorption capacity of at least about 4 g/g.

9. The fibers of claim 7, wherein the average degree of sulfate substitution is from about 0.1 to about 2.0.

10. The fibers of claim 7, wherein the average degree of sulfate substitution is from about 0.2 to about 1.0.

11. The fibers of claim 7, wherein the average degree of sulfate substitution is from about 0.3 to about 0.5.

12. The fibers of claim 7, wherein the cellulosic fibers are wood pulp fibers.

13. The fibers of claim 7, wherein the fibers are crosslinked with a crosslinking agent selected from the group consisting of a urea-based crosslinking agent, a polycarboxylic acid crosslinking agent, an aldehyde crosslinking agent, a dialdehyde crosslinking agent, and mixtures thereof.

14. The fibers of claim 13, wherein the crosslinking agent is applied to the fibers in an amount from about 0.01 to about 8.0 percent by weight based on the total weight of fibers.

15. The fibers of claim 13, wherein the crosslinking agent is applied to the fibers in an amount from about 0.02 to about 5.0 percent by weight based on the total weight of fibers.

16. A rollgood comprising the fibers of claims 1 or 7.

17. The rollgood of claim 16 further comprising another fiber.

18. The rollgood of claim 17, wherein the other fiber is at least one of fluff pulp fibers, crosslinked cellulosic fibers, cotton fibers, CTMP fibers, and synthetic fibers.

19. The rollgood of claim 16 further comprising an absorbent material.

20. The rollgood of claim 16 further comprising a binder material.

21. An absorbent article comprising the rollgood of claim 16.

22. An absorbent composite comprising the fibers of claims 1 or 7.

23. The composite of claim 22 further comprising another fiber.

24. The composite of claim 23, wherein the other fiber is at least one of fluff pulp fibers, crosslinked cellulosic fibers, cotton fibers, CTMP fibers, and synthetic fibers.

25. An absorbent article comprising the fibers of claims 1 or 7.

26. The article of claim 25, wherein the article is at least one of an infant diaper, an adult incontinence product, and a feminine care product.

27. An absorbent article comprising a liquid pervious topsheet, a liquid impervious backsheet attached to the topsheet, and an absorbent member intermediate the topsheet and backsheet, wherein the absorbent member comprises the fibers of claims 1 or 7.

28. An absorbent article comprising a liquid pervious topsheet, a liquid impervious backsheet attached to the topsheet, and an absorbent member intermediate the topsheet and backsheet, wherein the absorbent member comprises the rollgood of claim 16.

29. A method for making cellulosic fibers, comprising crosslinking sulfated cellulosic fibers to render the fibers substantially water-insoluble.

30. The method of claim 29, wherein crosslinking sulfated cellulosic fibers comprises treating sulfated cellulosic fibers with an amount of a crosslinking agent sufficient to render the crosslinked fibers substantially water insoluble.

31. The method of claim 30, wherein the amount of crosslinking agent ranges from about 0.01 to about 8.0 percent by weight crosslinking agent based on the total weight of fibers.

32. The method of claim 30, wherein the crosslinking agent is selected from the group consisting of a urea-based crosslinking agent, a polycarboxylic acid crosslinking agent, an aldehyde crosslinking agent, a dialdehyde crosslinking agent, and mixtures thereof.

33. The method of claim 30, wherein the crosslinking agent is applied to the fibers as an aqueous alcoholic solution.

34. The method of claim 29, wherein the sulfated cellulosic fibers have an average degree of sulfate substitution of from about 0.1 to about 2.0.

35. The method of claim 29 further comprising baling the crosslinked, sulfated fibers.

36. The method of claim 29 further comprising forming the crosslinked, sulfated fibers into a rollgood.

37. A method for making cellulosic fibers, comprising the steps of:
reacting cellulosic fibers with a sulfating agent to provide sulfated fibers;
applying a crosslinking agent to the sulfated fibers; and
curing the crosslinking agent to provide crosslinked, sulfated cellulosic fibers.

38. The method of claim 37, wherein the sulfating agent comprises sulfuric acid.

39. The method of claim 37, wherein the sulfating agent comprises a solution of sulfuric acid in an organic solvent.

40. The method of claim 37, wherein the organic solvent is an alcohol is selected from the group consisting of isopropanol, propanol, and butanol.

41. The method of claim 40, wherein the ratio of sulfuric acid to alcohol is about 2.4:1 by mole.

42. The method of claim 37 further comprising treating the sulfated fibers with a neutralizing agent prior to crosslinking.

43. The method of claim 42, wherein the neutralizing agent comprises a base.

44. The method of claim 42, wherein the neutralizing agent comprises a multivalent metal salt.

45. The method of claim 44, wherein the metal salt is selected from the group consisting of cerium nitrate, magnesium sulfate, and aluminum sulfate.

46. The method of claim 37, wherein the crosslinking agent is selected from the group consisting of a urea-based crosslinking agent, a polycarboxylic acid crosslinking agent, an aldehyde crosslinking agent, a dialdehyde crosslinking agent, and mixtures thereof.

47. The method of claim 37, wherein the crosslinking agent is applied to the fibers as an aqueous alcoholic solution.

48. The method of claim 37, wherein the cellulosic fibers are reacted with the sulfating agent at a temperature of about 4° C.

49. The method of claim 37 further comprising swelling the fibers prior to reacting the fibers with the sulfating agent.

50. The method of claim 49, wherein swelling the fibers comprises treating the fibers with a swelling agent selected from the group consisting of acetic acid, acetic anhydride, and mixtures thereof.

51. The method of claim 50 further comprising removing excess swelling agent prior to reacting the fibers with the sulfating agent.

52. The method of claim 37, wherein reacting the fibers with a sulfating agent comprises adding the fibers to an alcoholic solution of the sulfating agent.

53. The method of claim 52, wherein the fibers are cooled to about 4° C. prior to adding the fibers to the alcoholic solution of the sulfating agent.

54. The method of claim 52, wherein the alcoholic solution of the sulfating agent is cooled to about 4° C. prior to the addition.

55. The method of claim 37, wherein the sulfating agent is reacted with the fibers at a temperature of about 4° C.

56. The method of claim 42 further comprising separating the sulfated fibers from excess sulfating agent prior to treating the sulfated fibers with the neutralizing agent.

57. The method of claim 42 further comprising washing the sulfated fibers with alcohol solution prior to treating the sulfated fibers with the neutralizing agent.

58. The method of claim 37, wherein the cellulosic fibers further comprise magnesium sulfate.

59. The method of claim 37, wherein the sulfating agent further comprises magnesium sulfate.

60. The method of claim 40, wherein the alcoholic solution of the sulfating agent further comprises magnesium sulfate.

61. A method for making cellulosic fibers, comprising the steps of:
swelling dry cellulosic fibers with a swelling agent to provide swelled fibers;
separating excess swelling agent from the swelled fibers;
reacting swelled cellulosic fibers with a sulfating agent to provide sulfated fibers;
separating excess sulfating agent from the sulfated fibers;
treating the sulfated fibers with a neutralizing agent to provide fibers suitable for crosslinking;
applying a crosslinking agent to the sulfated fibers; and
curing the crosslinking agent to provide crosslinked, sulfated cellulosic fibers.

62. The method of claim 61, wherein the dry cellulosic fibers comprise never-dried cellulosic fibers solvent exchanged with an alcohol.

63. The method of claim 61, wherein the swelling agent is selected from the group consisting of acetic acid, acetic anhydride, and mixtures thereof.

64. The method of claim 61, wherein the sulfating agent comprises sulfuric acid.

65. The method of claim 61, wherein the sulfating agent comprises a solution of sulfuric acid in an alcohol.

66. The method of claim 65, wherein the ratio of sulfuric acid to alcohol is about 2.4:1.

67. The method of claim 65, wherein the alcohol comprises isopropanol.

68. The method of claim 61, wherein the neutralizing agent comprises sodium hydroxide.

69. The method of claim 61, wherein the crosslinking agent is applied to the fibers as an aqueous alcoholic solution.

70. The method of claim 61, wherein the cellulosic fibers are reacted with the sulfating agent at a temperature of about 4° C.

71. The method of claim 61, wherein the cellulosic fibers are reacted with the sulfating agent for a period of time from about 10 to about 60 minutes.

72. The method of claim 61 further comprising washing the sulfated fibers with an alcohol solution prior to treating the sulfated fibers with the neutralizing agent.

73. The method of claim 61, wherein the sulfating agent further comprises magnesium sulfate.

74. The product obtainable by the process of claim 29.

75. The product obtainable by the process of claim 37.

76. The product obtainable by the process of claim 61.

77. An absorbent core for an absorbent article, comprising a sulfated cellulosic fiber crosslinked to an extent to render the fiber substantially insoluble in water, wherein the core has a liquid absorption capacity of at least about 22 g/g.

78. An absorbent core for an absorbent article, comprising individual, water-swellable, water-insoluble, intrafiber crosslinked, sulfated cellulosic fibers, wherein the core has a liquid absorption capacity of at least about 22 g/g.
